(19) United States
(12) Patent Application Publication
(10) Pub. No.: US 2026/0258347 A1
(43) Pub. Date: Sep. 3, 2026

(54) Bioink Compositions for Producing Cell-Laden Hydrogel Constructs and Methods of Use

(71) Applicant: Emory University, Atlanta, GA (US)

(72) Inventors: Vahid Serpooshan, Atlanta, GA (US); Holly Bauser-Heaton, Atlanta, GA (US); Martin L. Tomov, Atlanta, GA (US)

(21) Appl. No.: 18/994,591

(22) PCT Filed: Jul. 14, 2023
(86) PCT No.: PCT/US2023/027765

§ 371 Date: Jan. 14, 2025

Related U.S. Application Data
(60) Provisional application No. 63/389,380, filed on Jul. 15, 2022.

(51) Int. Cl. C12N 5/00

(52) U.S. Cl. CPC C12N 5/0068; C12N 2533/30; C12N 2533/54; C12N 2533/90

(57) ABSTRACT

This disclosure relates to bioink compositions for the fabrication of hydrogels or cell-laden hydrogel constructs comprising methacrylated polyethylene glycol and methacrylated gelatin. In certain embodiments, this disclosure relates to methods of fabricating cell-laden hydrogel constructs using bioink composition disclosed herein. In certain embodiments, cells are introduced into the hydrogel scaffold providing a cell-laden hydrogel construct.

### CROSS-REFERENCE TO RELATED APPLICATIONS

This application claims the benefit of U.S. Provisional Application No. 63/389,380 filed Jul. 15, 2022. The entirety of this application is hereby incorporated by reference for all purposes.

### STATEMENT REGARDING FEDERALLY SPONSORED RESEARCH OR DEVELOPMENT

This invention was made with government support under HL27925 awarded by the National Institutes of Health. The government has certain rights in the invention.

### BACKGROUND

Organ failure is a common cause of death. Artificial materials that can be implanted or integrated with living tissue are contemplated in therapies. Biodegradable hydrogels can trap large amounts of water making them attractive candidates as biomimetics of natural environments. Three-dimensional (3D) hydrogels can be generated using digital light processing (DLP) bioprinters. These devices contain movable platforms and/or controllable mirrors that rotate projecting the path of light onto a photosensitive material for fabricating specific shapes. Lasers or ultraviolet light are capable of initiating polymerization of liquid materials which can be arranged in pre-designed 3D structures, e.g., through layer-by-layer construction. However, generating bioprinted hydrogel cell-laden constructs with adequate cellular integrity and long-term structural fidelity has been challenging. Thus, there is a need to identify improved materials.

Miller et al. report bioactive hydrogels made from step-growth derived polyethylene glycol peptide macromers. Biomaterials, 2010, 31(13): 3736-3743

Van Hove et al. report microwave-assisted functionalization of poly(ethylene glycol) and on-resin peptides for use in hydrogel formation. J Vis Exp, 2013, (80), e50890. See also PCT Publication WO2015021044.

Ning et a. report embedded 3D bioprinting of gelatin methacryloyl-based constructs. ACS Appl Mater Interfaces, 2020, 12, 40, 44563-4457.

Bandyopadhyay et al. report 3D bioprinting of photo-crosslinkable silk methacrylate (SilMA)-polyethylene glycol diacrylate (PEGDA) bioink for cartilage tissue engineering. J Biomed Mater Res, 2022, 110:884-898.

Martinez et al. report double network bioinks. PCT Publication WO2020077118. See also Olga et al. PCT Publication WO2020081982.

References cited herein are not an admission of prior art.

### SUMMARY

This disclosure relates to bioink compositions for the fabrication of hydrogels or cell-laden hydrogel constructs comprising a photoinitiator, methacrylated-polyethylene glycol, and methacrylated gelatin and/or other methacrylated component(s). In certain embodiments, this disclosure relates to methods of fabricating cell-laden hydrogel constructs using bioink composition disclosed herein comprising the steps of providing a liquid solution comprising a photoinitiator, methacrylated polyethylene glycol, methacrylated gelatin and/or another methacrylated component; and exposing a zone within the liquid solution with light under conditions that polymerization provides a hydrogel scaffold within the zone. In certain embodiments, cells are contained within the liquid solution or cells are introduced into the hydrogel scaffold providing a cell-laden hydrogel construct.

In certain embodiments, the methacrylated-polyethylene glycol is 90-95% functionalized. In certain embodiments, the construct further comprises cells. In certain embodiments, the methacrylated gelatin is 60-75% functionalized. In certain embodiments, this disclosure relates to methods of making three-dimensional hydrogel structures comprising cells using compositions disclosed herein. In certain embodiments, the bioink is a liquid at around room temperature. In certain embodiments, the bioink is a liquid at about 4 degrees Celsius.

In certain embodiments, the gelatin is fish (cold-water) gelatin. In certain embodiments, the gelatin is porcine gelatin. In certain embodiments, the gelatin is fish (cold-water, cold-water fish skin, teleostean fish) gelatin optionally further comprising porcine gelatin. In certain embodiments, the methacrylated gelatin is made by the process of contacting a gelatin with a methacrylating agent. In certain embodiments, the methacrylating agent is methacrylic anhydride.

In certain embodiments, the methacrylated-polyethylene glycol has an average molecular weight of between 1,000 Da and 10,000 Da 1,000 and 20,000 Da, 1,000 and 30,000 Da, 3,000 and 20,000 Da, and 1,000 and 100,000 Da. In certain embodiments, the methacrylated-polyethylene glycol made by the process of contacting polyethylene glycol with a methacrylating agent. In certain embodiments, the methacrylating agent is methacrylic anhydride.

In certain embodiments, the methacrylated polyethylene glycol has a molecular weight of between 3,000 and 100,000 Da.

In certain embodiments, the methacrylated polyethylene glycol has a molecular weight of between 15,000 and 400,000 Da.

In certain embodiments, the composition is a liquid solution further comprising phosphate buffered saline.

In certain embodiments, the methacrylated gelatin is between 20-5% by weight to volume of the liquid.

In certain embodiments, the photoinitiator is lithium phenyl-2,4,6-trimethylbenzoylphosphinate (LAP). In certain embodiments, the composition further comprises tartrazine. In certain embodiments, the ratio of LAP to tartrazine is between 0.5-1% by weight.

In certain embodiments, the composition further comprises tartrazine. In certain embodiments, the ratio of LAP to tartrazine is between 0.25-1% by weight.

In certain embodiments, constructs and materials disclosed herein are biodegradable and biocompatible. In certain embodiments, the bioink solutions or hydrogels derived therefrom contain cells and agents commonly found in a growth medium. In certain embodiments, cells within the cell-laden constructions are epithelial cells. In certain embodiments, cells within the cell-laden constructions are capable of replicating or surviving for more than 10 days, 15 days, 20 days, or 30 days. In certain embodiments, cells within the cell-laden constructions are capable of replicating or surviving for more than 60 days.

### BRIEF DESCRIPTION OF THE SEVERAL VIEWS OF THE DRAWINGS

FIG. 1 shows lumen X bioprinted constructs fixed and stained for DAPI, CD31, and α-Tubulin markers: Preliminary evaluation suggests that cells can spread out about 300 μm from an interface. Bioprinted constructs can support embedded cells at east for 14 days post-printing. Endothelial cell specific markers (CD31 and VWF) appear to be upregulated in 3D.

FIG. 2 shows a table of example bioinks tested. Experiments indicate the following was a desirable composition: 10% gelMA (w/v)—porcine OR cold-water fish 5-10% gelatin (w/v)—cold water fish—5-2.5% PEGDM6000 (w/v)—0.5% LAP (w/v), and 1.5 mM tartrazine (Generation 2 Bioink for DLP).

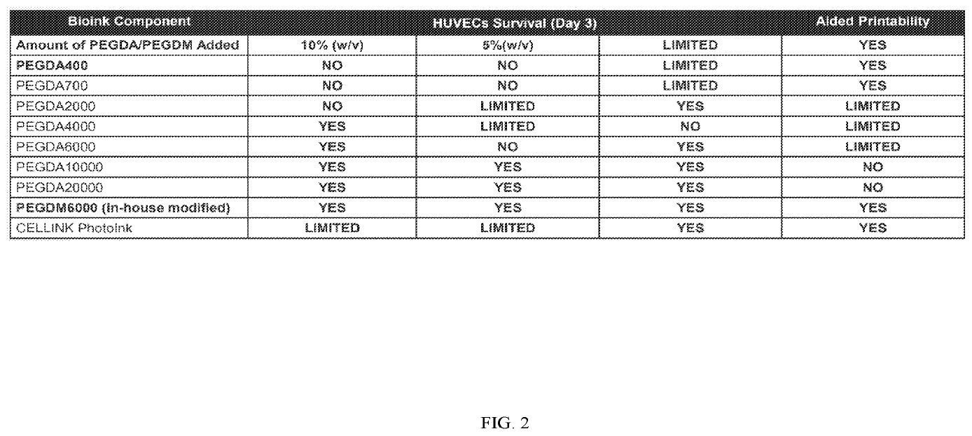

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

“About” as used herein when referring to a measurable value such as an amount, a temporal duration, and the like, is meant to encompass variations of ±20% or ±10%, or ±5%, or ±1%, or ±0.1% from the specified value, as such variations are appropriate to perform the disclosed methods.

“Methacrylate” refers to the compound with the chemical formula CH₂═C(CH₃)(C═O)OCH₃ and a “methacrylated” compound refers to derivatives, e.g., ester amide, thiol, or derivatives, containing the chemical formula CH₂═C(CH₃)(C═O)X wherein X is the derivative linking group. “Acrylate” refers to the compound with the chemical formula CH₂═CH(C═O)OCH₃ and an “acrylated” compound refers to derivatives, e.g., ester, amide, or thiol derivatives, containing the chemical formula CH₂═CH(C═O)X wherein X is the derivative linking group. It is appreciated that certain light exposure of terminal methacrylic and acrylic groups will polymerize, thus conjugate through covalent chemical bonds, in the presence of a photoinitiator.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids such as histidine, isoleucine, leucine, lysine, methionine, phenylalanine, threonine, tryptophan, valine, and others; vitamins such as retinol, carotene, thiamine, riboflavin, niacin, biotin, folate, and ascorbic acid; a carbohydrate such as glucose, galactose, fructose, or maltose; inorganic salts such as sodium, calcium, iron, potassium, magnesium, zinc; serum; and buffering agents. Additionally, a growth medium may contain a pH indicator, e.g., phenol red. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate.

Various growth mediums are known in the art. For example, Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8). Dulbecco's modified Eagle's medium (DMEM) is a growth medium which contains additional components such as glycine, serine, and ferric nitrate with increased amounts of vitamins, amino acids, and glucose. Animal serum such as fetal bovine serum (FBS) is sometimes added to a growth media as a supplement.

As used herein, the term “gel” refers to a three-dimensional polymeric structure that itself is insoluble in a particular liquid, but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “hydrogel” will be used throughout this application to refer both to polymeric structures that have absorbed water and to polymeric structures that have absorbed a liquid other than water, it being readily apparent to those skilled in the art from the context whether the polymeric structure is simply a “gel” or a “hydrogel.”

As used herein, the term “biodegradable” in reference to a material refers to a molecular arrangement in the material that when implanted to a subject, e.g., human, will be broken down by biological mechanism such that a decomposition of the molecular arrangement will occur and the molecular arrangement will not persist for over a long period of time, e.g., the molecular arrangement will be broken down by the body after a several days or a couple weeks. In certain embodiments, the disclosure contemplates that the biodegradable material will not exist after a month or several months.

As used herein, “biocompatible” refers to any material, which when implanted in a mammal, does not typically provoke an adverse response in the mammal. A biocompatible material, when introduced into an individual, is typically not toxic or injurious to that individual, nor does it induce immunological rejection of the material in the mammal.

The terms “drug,” “agent,” “pharmaceutical agent,” and similar terms are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect. Examples include analgesics, steroidal anti-inflammatoir steroidal anti-inflammatoires, statins, antibiotics, anti-neoplastics, anti-spasmodics, modulators of cell-extracellular matrix interactions, proteins, hormones, enzymes and enzyme inhibitors, anticoagulants and/or antithrombic agents, and vasodilating agents.

### Bioink Compositions and Methods of Use

Digital Light Processing (DLP) printers typically have a light source, curing plane (e.g., translucent bottom), fabricated object, and build plate (e.g., solid platform). The solid platform is typically capable of being pulled up in the z-axis. A vat with a translucent bottom is filled with liquid ink (bioink) on the x-and y-axis. A projector focuses light at the translucent bottom plate inducing polymerization. The polymerized material to adheres/sticks/integrates into the solid platform. Moving the solid platform up in in the z-axis causes the bioink to fill in gaps created between the polymerized material attached to solid platform and the translucent plate. Repeated light induced polymerization allows for layer-by-layer printing, making it possible to print complex user-defined microstructures. Bioinks used in the fabrication process are ideally non-cytotoxic and possess low viscosity during printing.

In certain embodiments, bioink constructs disclosed herein have specific rheological properties to allow them to be used in bioprinting applications on digital light processing (DLP) bioprinters. In certain embodiments, this disclosure relates to bioink compositions for the fabrication of hydrogel constructs comprising a photoinitiator, methacrylated polyethylene glycol, and methacrylated gelatin and optionally another methacrylate component. In certain embodiments, this disclosure relates to methods of fabricating cell-laden hydrogel constructs using bioink composition disclosed herein comprising the steps of providing a liquid solution comprising, a photoinitiator, dimethacrylated polyethylene glycol, methacrylated gelatin, and optionally cells; and exposing a zone within the liquid solution with light under conditions that polymerization provides a hydrogel construct or cell-laden hydrogel construct within the zone. In certain embodiments, the bioink is a liquid at room temperature. In certain embodiments, the bioink is a liquid at 4 degrees Celsius.

In certain embodiments, this disclosure relates to bioink compositions for the fabrication of cell-laden hydrogel constructs comprising a liquid/aqueous pH buffered solution with a photoinitiator; methacrylated-polyethylene glycol, methacrylated gelatin and cells. In certain embodiments, this disclosure relates to methods of fabricating cell-laden hydrogel constructs using bioink composition disclosed herein comprising the steps of providing a liquid solution of cells optionally comprising, a photoinitiator, dimethacrylated polyethylene glycol, and methacrylated gelatin; and exposing a zone within the liquid solution with light under conditions that polymerization provides a cell-laden hydrogel construct within the zone.

In certain embodiments, the bioink solutions include methacrylated gelatin (gelMA), from porcine, bovine, or cold-water fish source, that is supplemented with pore-inducing agents, such as unmodified gelatin (cold water fish, porcine, bovine), alginate and alginate-methacrylate, hyaluronic acid and hyaluronic acid methacrylate, silk fibroin and silk fibroin methacrylate, thiolated gelatin and silk fibroin, norbornene-modified gelatin and silk fibroin, or fibrin and collagen, to enhance mass transport properties of fabricated tissue constructs.

In certain embodiments, bioink solutions comprise biologically active reagents to enhance cell viability and function in the bioprinted constructs, such as gelatin, cellulose, fibrin, collagen, laminin, glucomannan, alginate, hyaluronic acid, silk, and fibronectin. In certain embodiments, each of these components may be incorporated into a hydrogel scaffold by being methacrylated, e.g., providing methacrylated gelatin, methacrylated cellulose, methacrylated fibrin, methacrylated collagen, methacrylated laminin, methacrylated glucomannan, methacrylated alginate, methacrylated hyaluronic acid, methacrylated silk, methacrylated fibronectin, and combinations thereof.

In certain embodiments, the bioink solutions include methacrylated gelatin (gelMA), from porcine or cold-water fish source, that is supplemented with pore-inducing agents, such as unmodified gelatin (cold water fish) or fibrin, to enhance mass transport properties of fabricated tissue constructs.

In certain embodiments, bioink solutions comprise biologically active reagents to enhance cell viability and function in the bioprinted constructs such as gelatin, cellulose, fibrin, collagen, laminin, glucomannan, alginate and fibronectin. In certain embodiments, each of these components may be incorporated into a hydrogel scaffold by being methacrylated, e.g., providing methacrylated gelatin, methacrylated cellulose, methacrylated fibrin, methacrylated collagen, methacrylated laminin, methacrylated glucomannan, methacrylated alginate, methacrylated fibronectin, and combinations thereof.

In certain embodiments, bioink solutions comprise methacrylated poly(ethylene glycol) of various molecular weights. The certain embodiments, methacrylated polyethylene glycol is polymethacrylated-or dimethacrylated-polyethylene glycol which is linear, branched, 4-arm, 8-arm, hyperbranched, or mixtures thereof. In certain embodiments, methacrylated poly(ethylene glycol) if functionalized with/conjugated to a fluorescent dye.

In certain embodiments, bioink solutions comprise an ultraviolet (UV), blue light, or visible light photoinitiator and optionally a same-wavelength photo-absorbing reagent. In certain embodiments, the photoinitiator is lithium phenyl-2,4,6-trimethylbenzoylphosphinate (LAP), 2-hydroxy-4′-(2-hydroxyethoxy)-2-methylpropiophenone (IRGACURE™-2959), water-soluble Ruthenium, and/or Eosin-Y.

In certain embodiments, bioink solutions comprise functional nanoparticles, such as superparamagnetic iron oxide nanoparticles (SPIONs), gold nanoparticles and nanorods, and gadolinium oxide nanoparticles, to confer specific biological function to the developed bioink, such as enhanced antibacterial properties, electrical conductivity, and imaging properties.

In certain embodiments, bioink formulations are application dependent, e.g., presented bioink formulations can be printed at high resolution (e.g., 25×25 μm, between 10-50 μm×10-50 μm, or more in a two dimensional XY direction and between 10-100 or 25-100 μm or more in a three dimensional Z direction) and are supportive of cellularization with a range of cells, such as immortalized cancer cell lines, hepatocytes, primary endothelial and smooth muscle cells, as well as stem cells derived from tissue-specific populations, such as cardiomyocytes and neurons.

In certain embodiments, cells survive withing the hydrogel for more than two or three weeks, or one or two months, post bioprinting and exhibit normal function.

In certain embodiments, bioink formulations cover high resolution bioprinting of small-to very large-scale models for biomedical research, as well as clinical and industrial applications.

In certain embodiments, bioink formulations further provide for tissue-specific peptides and bioactive molecules to be stably incorporated within the bioink, to tailor the final formulation to a specific tissue target.

In certain embodiments, this disclosure relates to bioink compositions for the fabrication of hydrogel constructs or cell-laden hydrogel constructs comprising photoinitiator; methacrylated-polyethylene glycol, and methacrylated gelatin. In certain embodiments, this disclosure relates to methods of fabricating cell-laden hydrogel constructs using bioink composition disclosed herein comprising the steps of providing a liquid solution comprising a photoinitiator, methacrylated polyethylene glycol, and methacrylated gelatin; and exposing a zone within the liquid solution with light under conditions that polymerization provides a hydrogel scaffold within the zone.

In certain embodiments, this disclosure relates to methods of fabricating cell-laden hydrogel constructs using bioink composition disclosed herein comprising the steps of providing a pH buffered liquid aqueous solution of cells comprising, a photoinitiator, methacrylated-polyethylene glycol, methacrylated gelatin, and exposing a zone within the liquid solution with light under conditions that polymerization provides a cell-laden hydrogel construct within the zone.

In certain embodiments, the bioink solutions include methacrylated gelatin (gelMA), from porcine, bovine, or cold-water fish source, that is supplemented with pore-inducing agents, such as unmodified gelatin (cold water fish, porcine, bovine), alginate and alginate-methacrylate, hyaluronic acid and hyaluronic acid methacrylate, silk fibroin and silk fibroin methacrylate, thiolated gelatin and silk fibroin, norbornene-modified gelatin and silk fibroin, or fibrin and collagen, to enhance mass transport properties of fabricated tissue constructs.

In certain embodiments, the methacrylated-polyethylene glycol is 90-95% functionalized. In certain embodiments, the construct further comprises cells. In certain embodiments, the methacrylated gelatin is 60-75% functionalized. In certain embodiments, this disclosure relates to methods of making three-dimensional hydrogel structures comprising cells using compositions disclosed herein.

In certain embodiments, the methacrylated-polyethylene glycol is 90-95% functionalized as determined by proton NMR analysis from the observed ratio of terminal methacrylated protons to central polyethylene glycol protons. In certain embodiments, the methacrylated gelatin is 60-75% functionalized.

In certain embodiments, the methacrylated gelatin is 60-75% functionalized as determined by proton NMR analysis from the observed ratio of terminal methacrylated protons to unmodified protons. In certain embodiments, the peak area of aromatic acids in the samples of synthesized gelMA compared to unmodified gelatin was employed as a reference in each NMR spectrum, and the degree of methacrylation (DM) can be calculated based on changes in the peak areas corresponding to lysine methylene protons around about 3.0 ppm.

In certain embodiments, the gelatin is cold-water fish gelatin. In certain embodiments, the gelatin is porcine gelatin. In certain embodiments, the gelatin is cold-water fish gelatin optionally further comprising porcine gelatin. In certain embodiments, the methacrylated gelatin made by the process of contacting a gelatin with a methacrylating agent. In certain embodiments, the methacrylating agent is methacrylic anhydride.

In certain embodiments, the methacrylated-polyethylene glycol has an average molecular weight of between 3,000 and 100,000 Da. In certain embodiments, the methacrylated-polyethylene glycol has an average molecular weight of between 3,000 and 6,000 Da, or between 5,000 and 7,000 Da.

In certain embodiments, the methacrylated-polyethylene glycol has an average molecular weight of between 3,000 and 20,000 Da. In certain embodiments, the methacrylated-polyethylene glycol has an average molecular weight of between 3,000 and 6,000 Da, or between 5,000 and 7,000 Da.

In certain embodiments, the methacrylated-polyethylene glycol made by the process of contacting polyethylene glycol with a methacrylating agent. In certain embodiments, the methacrylating agent is methacrylic anhydride.

In certain embodiments, the methacrylated-gelatin is between 20-5% by weight to volume of the liquid.

In certain embodiments, the photoinitiator is lithium phenyl-2,4,6-trimethylbenzoylphosphinate (LAP). In certain embodiments, the composition further comprises tartrazine. In certain embodiments, the ratio of LAP to tartrazine is between 0.25-1% by weight.

In certain embodiments, gelatin, either porcine or cold-water fish, is methacrylate to 65-25 70% of all available sites. In certain embodiments, polyethylene glycol (MW 400-100,000 Da) is methacrylated to 90-95% of available modification sites. In certain embodiments, LAP and tartrazine are used together. In certain embodiments, ingredients are suspended in a phosphate buffered solution (PBS), which is then filtered and sterilized.

In certain embodiments, the photoinitiator is lithium phenyl-2,4,6-trimethylbenzoylphosphinate (LAP). In certain embodiments, the composition further comprises tartrazine. In certain embodiments, the ratio of LAP to tartrazine is between 0.5-1% by weight, or 0.25-1% by weight.

In certain embodiments, gelatin, either porcine or cold-water fish, is methacrylate to 65-70% of all available sites. In certain embodiments, polyethylene glycol (400-20,000 Da) is methacrylate to 90-95% of available modification sites. In certain embodiments, LAP and tartrazine are used together. In certain embodiments, ingredients are suspended in a phosphate buffered solution (PBS), which is then filtered and sterilized.

In certain embodiments, methacrylation of the gelMA is around 65-70% in order to improve cell survivability and attachment post-printing. In certain embodiments, modification by methacrylation of sites on gelMA allow for functional peptides and small bioactive molecules to be anchored. In certain embodiments, compositions and constructs do not contain acrylated polyethylene glycol. In certain embodiments, methacrylated polyethene glycol and methacrylated gelatin generates softer and more porous gels once crosslinked, compared to compositions and constructs produced using acrylated polyethylene glycol and methacrylated gelatin. In certain embodiments, the bioink is more compatible with seeding or encapsulating cells, e.g., allowing for survival of the cells for more than 5 days, 10 days, two weeks, or indefinitely.

In certain embodiments, the bioink composition comprises a mix of cold-water fish methacrylated gelatin and porcine methacrylated gelatin, plus cold-water fish gelatin, and high molecular weight (3000-20,000 Da) methacrylated polyethene glycol. In certain embodiments, the bioink composition further comprises LAP and tartrazine to specific ratios (0.5-1%) based on application. In certain embodiments, the bioink composition is a liquid at 4 degrees Celsius and at room temperature, thus increasing the lifetime of the bioink where it is printable and also adding enough porosity that encapsulated cells survive and remodel in the cell laden constructs more effectively and retaining the ability to print in high resolution.

In certain embodiments, the bioink composition comprises a mix of cold-water fish methacrylated gelatin and porcine methacrylated gelatin, plus cold-water fish gelatin, and high molecular weight (3000-100,000 Da) methacrylated polyethene glycol. In certain embodiments, the bioink composition further comprises LAP and tartrazine to specific ratios (0.5-1% or 0.25-1%) based on the application. In certain embodiments, the bioink composition is a liquid at 4 degrees Celsius and at room temperature, thus increasing the lifetime of the bioink where it is printable and also adding enough porosity so that encapsulated cells survive and remodel in the cell laden constructs more effectively and retaining the ability to print in high resolution.

In certain embodiments, methacrylation of gelatin to 65-70% is performed by incubating the gelatin and methacrylate for a time duration of about or less than 2 hours or 2.5 hours at room temperature providing methacrylated gelatin (gelMA). In certain embodiments, the incubation is in an aqueous solution. In certain embodiments, the incubation is in a pH buffered aqueous solution, e.g., pH between 6 and 8. In certain embodiments, the product produce after incubation is frozen, placed under a vacuum, and allowed to warm to room temperature providing a lyophilized gelMA. In certain embodiments, the pH of the solution of gelMA is adjusted to about 7.5 (e.g. between 7.0 to 8.0) before lyophilization. In certain embodiments, lyophilized gelMA is reconstituted in a liquid aqueous solution adjusted to about 7.5 (e.g. between 7.0 to 8.0).

In certain embodiments, the reconstituted bioink composition comprises LAP and tartrazine. In certain embodiments, the concentration of methacrylated gelatin is about 10% (w/v) aqueous solution e.g., between 5% and 15%, or between 7% and 13%, or between 9% and 11%. In certain embodiment, the aqueous solution is sterilized. In certain embodiment, the aqueous solution comprises a phosphate buffered solution (PBS), which has use as a base bioink solution. In certain embodiment, the base bioink solution has about 1% (w/v) of LAP. In certain embodiment, the base bioink solution with LAP has a pH of about 7.5 (between 7.0 and 8.0). In certain embodiment, the base bioink solution with LAP comprises about 1.5 mM tartrazine added to the solution, which is filter sterilized.

In certain embodiments, the bioink composition comprises methacrylated polyethylene glycol. In certain embodiments, the methacrylated polyethylene glycol has an average molecular weight of 1,000 to 3,000 Da, 3,000 to 6,000 Da, and 6,000 to 10,000 Da, and/or 10,000 to 20,000 Da).

In certain embodiments, the bioink composition comprises methacrylated polyethylene glycol. In certain embodiments, the methacrylated polyethylene glycol has an average molecular weight of 1,000 to 3,000 Da, 3,000 to 6,000 Da, and 6,000 to 10,000 Da, and 10,000 to 20,000 Da, and/or 20,000 to 100,000 Da.

In certain embodiments, the bioink composition further comprises a drug, e.g., an antibiotic agent, or mixture of drugs or antibiotic agents. In certain embodiments, the antibiotic agent is antimycotic, i.e., a mixture of penicillin, streptomycin, and amphotericin B.

In certain embodiments, this disclosure relates to methods for fabricating a cell laden construct using three-dimensional bioprinting. In certain embodiments, the method comprises preparing a bioink composition as disclosed herein, wherein the bioink is prepared by mixing components disclosed herein and exposing the bioink composition to a light source e.g., laser, light/ultraviolet light, forming a scaffold. In certain embodiments, the scaffold is polymerized for the predetermined time, and the scaffold is optionally washed with a solution e.g., phosphate buffered saline (PBS).

In certain embodiments, a first layer is formed by directing light to a specific zone within the bioink causing the formation of a polymerized hydrogel in the zone and moving the direction of light to an adjacent zone for formation of a second layer in the adjacent zone. In certain embodiments, a first layer is formed by directing light to a specific zone within the bioink causing the formation of a hydrogel and moving the hydrogel so that light will be directed to an adjacent zone that has not yet formed a polymerized hydrogel forming a second layer.

In certain embodiments, the polymerized hydrogel is lyophilized to remove water. In certain embodiments, the lyophilized hydrogel is exposed to cells and a growth medium for migration of the cells and growth medium ingredients into the polymerized hydrogel scaffold.

In certain embodiments, the polymerized hydrogel is exposed to cells and a growth medium for migration of the cells and growth medium ingredients into the polymerized hydrogel scaffold. In certain embodiments, cells and a growth medium are pushed or migrate into the polymerized hydrogel scaffold by external pressure or capillary pressure of a flowing liquid comprising cells or other components through the scaffold.

In certain embodiments, this disclosure relates to methods for fabricating a cell laden construct using three-dimensional bioprinting. In certain embodiments, the method comprises preparing a bioink composition as disclosed herein, wherein the bioink is prepared by mixing components disclosed herein, and optionally cells, and optionally a growth medium and exposing the bioink composition to light/ultraviolet light to form a scaffold. In certain embodiments, the scaffold is polymerized for a predetermined time and the scaffold is optionally washed with a solution e.g., phosphate buffered saline (PBS).

In certain embodiments, the bioink disclosed herein may be introduced onto a print plate such as a petri plate, a quartz plate, or a glass slide. In certain embodiments, the print plate may be made of metals for example but not limited to aluminium and stainless steel. In certain embodiments, the bioink is on the print plate for layer-by-layer construction of the scaffold after repeated exposure to light/ultraviolet light.

In certain embodiments, the cells may be derived from epithelial, muscular, nervous, or connective tissue. In certain embodiments, the cells are obtained from healthy or diseased donor. In certain embodiments, the cells may be genetically engineered cells, including induced pluripotent stem cells (iPSCs) or disease specific model cells.

In certain embodiment, the tissue specific cells may be derived from a tissue selected from a group consisting of liver, gastrointestinal, pancreatic, kidney, lung, tracheal, vascular, skeletal muscle, cardiac, skin, smooth muscle, connective tissue, corneal, genitourinary, breast, reproductive, endothelial, epithelial, fibroblast, neural, Schwann, adipose, bone, bone marrow, pericytes, mesothelial, endocrine, stromal, lymph, and blood, i.e., cells derived therefrom.

In certain embodiment, the bioink or scaffold created therefrom as reported herein optionally comprises cells and a growth medium. In certain embodiment, the growth medium is serum such as bovine serum (cow), chicken serum, caprine (goat), equine (horse), human, ovine (sheep), porcine (pig) or rabbit sera.

In certain embodiment, the bioink reported herein comprises a growth medium of chemically defined supplements. In certain embodiment, the chemically defined supplements may include growth factors or growth hormones or growth regulating factors such as EGF, VEGF, hydrocortisone, insulin, epinephrine, transferrin, heparin, non-essential amino acids, PDGF, and/or TGF.

In certain embodiment, the bioink or scaffold created therefrom as reported herein optionally comprises a cryoprotectant or a combination of various cryoprotectants. In certain embodiment, the cryoprotectant is dimethyl sulfoxide (DMSO), glycerol, hydroxyethyl starch, polyethylene glycol, or combinations thereof.

### Methacrylated Gelatin (gelMA)

Mix gelatin, either porcine or cold-water fish, with methacrylic anhydride for a limited time or limited concentration provides methacrylated gelatin with only 65-70% of all available sites. Methacrylating less than all of the sites allows for modification of gelMA sites with additional functional peptides and small bioactive molecules.

### Polyethylene Glycol Dimethacrylate (PEGDM) of Certain Molecular Weights

Mixing polyethylene glycol with methacrylic anhydride provides dimethacrylated polyethylene glycol (PEGDM). Dissolve the PEGDM in minimal amount of dichloromethane. Dilute the PEGDM in 10-15 times in excess dimethylformamide (DMF) for 20 min. Place the PEGDM solution in an appropriate size exclusion dialysis tubing (1000 Da MWCO for PEGDM3000; 3000 Da MWCO for PEGDM6000; 10000 Da MWCO for PEGDM20000 and above, up to PEGDM 100,000). Dialyze against 10 times volume dd-water/filtered water (approximately 1-1.5 L) for 5 days, changing the water every day in the morning and in the afternoon. Adjust the pH of the dialyzed PEGDM to be about 7.5 and filter-sterilize the solution using a 0.22 μm PES filter. Transfer the filtered PEGDM to 50 ml conical tubes, filling them with no more than 35 mL solution, securely close the caps of the tubes and freeze the PEGDM solution at −80 C for at least 2 days. Place the frozen tubes in a lyophilizer and lyophilize for 5 days. Store the final PEGDM product at −20 to −80 C, protected from light.

### Bioink Solution

The base gelMA bioink solution was mixed (before Tartrazine is added) with 7.5% (w/v) of cold-water gelatin and add 15 mM PEGDM, pH the final solution to 7.5 and then add 1.5 mM tartrazine and 1× antibiotic-antimycotic to the bioink. Filter-sterilize the solution with 0.45 μm PES filter and store at 4 C for use, protected from light. The reconstituted bioink is generally best used within 4-6 weeks.

### Cellularized DLP/Lumen X Bioink Formulation Progress and Validation

### Trial 1 Experimental Setup

Bioink: 10% gelMA+5% gelatin+5% PEGDM6000+0.5% LAP+1.5 mM Tartrazine. Stained bioprinted constructs (Day 14) for α-Tubulin, DAPI, and CD31 markers.

### Trial 2 Experimental Setup

Bioink: 10% gelMA+5% gelatin+2.5% PEGDM6000+0.5% LAP+1.5 mM Tartrazine.

### Trial 3 Experimental Setup

Bioprinted the single conduit construct with ~10×10⁶ cells/ml seeded within the bioink.

Bioink 1: 10% gelMA+5% gelatin+2.5% PEGDM6000+0.5% LAP+1.5 mM Tartrazine.

Bioink 2: 10% gelMA+5% gelatin+10 mM PEGDA400+0.5% LAP+1.5 mM Tartrazine.

### Results

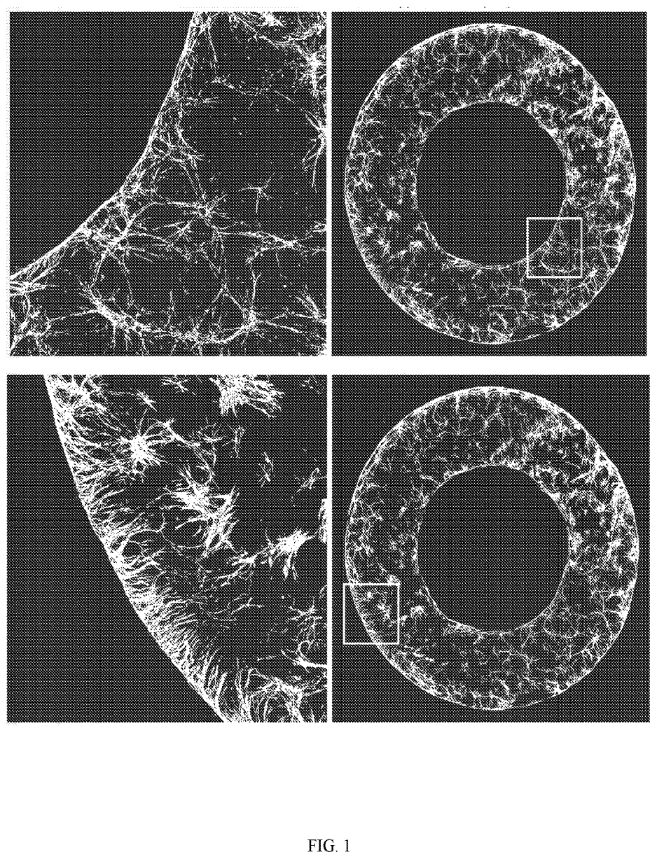

Live cell images for day 7 are shown in FIG. 1. Preliminary data suggests the depth that cells can tolerate within the bioprinted constructs is between 200-400 microns (based on cell spreading). Supplementing the bioink with fibrin/thrombin may improve pores. It is desirable to keep the methacrylation of the gelMA to around 65-70% to improve cell survivability and attachment post-printing. One can modify sites on the gelMA with functional peptides and small bioactive molecules. The process provides dimethacrylated PEG (PEGDM), rather than acrylated PEG. PEGDM generates softer and more porous gels once crosslinked, compared to PEGDA, thus it is more compatible with seeding or encapsulating cells.

Bioinks typically use only porcine gelMA, or porcine gelMA supplemented with low molecular weight (400 Da) PEGDA. Bioink tested herein utilized a mix of cold-water fish gelMA and porcine gelMA, plus cold-water fish gelatin, and high molecular weight (3000-20000 Da) PEGDM (in contrast to traditional PEGDA; methacrylate vs acrylate). The bioink was supplemented with LAP and tartrazine to specific ratios (0.5-1%) based on application. This formulation provides a liquid at 4 degrees C and room temperature, thus increasing the lifetime of the bioink where it is printable with enough porosity that encapsulated cells survive and remodel in the bioink more effectively. The methacrylation of gelMA of 65-70% is performed by incubating the reaction for a shorter period of time/specific time duration (2 hours vs 3.5 hours). The pH the gelMA is adjusted to about 7.5 before lyophilization.

To prepare the disclosed cell-encapsulating DLP bioinks (PEG MW 3000, 6000, 20000, and 100,000), PEG-dimethacrylate (PEGDM) was used (as opposed to PEG-diacrylate (PEGDA)). The base gelMA bioink solution was mixed before tartrazine is added with 7.5% (w/v) of cold-water gelatin and add 15 mM PEGDM, pH the final solution to 7.5 and then add 1.5 mM tartrazine and 1× antibiotic-antimycotic to the bioink. The solution is filter-sterilized with 0.45 μm PES filter and store at 4 C for use, protected from light. The reconstituted bioink is generally used within 4-6 weeks.

Additional contemplated bioink components include supplement of the bioink with fibrinogen at 1-10 mg/mL concentration, increase gelatin concentration to 10%, and decrease LAP concentration to 0.25%. One can supplement the bioink with alginate and/or hyaluronic acid. One can supplement the bioink with alginate methacrylate and/or hyaluronic acid methacrylate. One can supplement the bioink with thiolated and norbornene modified gelatin and/or alginate, and/or hyaluronic acid, and/or silk.

1. A composition for fabrication of cell-laden hydrogel constructs comprising
photoinitiator;
methacrylated polyethylene glycol; and
methacrylated gelatin.

2. The composition of claim 1, wherein the methacrylated-polyethylene glycol is 90-95% functionalized.

3. The composition of claim 1, wherein the methacrylated gelatin is 60-75% functionalized.

4. The composition of claim 1, wherein the gelatin is cold-water fish gelatin optionally further comprising porcine gelatin.

5. The composition of claim 1, wherein the composition is a liquid solution further comprising phosphate buffered saline.

6. The composition of claim 1, wherein the methacrylated gelatin is between 20-5% by weight to volume of the liquid.

7. The composition of claim 1, wherein the photoinitiator is lithium phenyl-2,4,6-trimethylbenzoylphosphinate (LAP).

8. The composition of claim 1, further comprising tartrazine.

9. The composition of claim 1, further comprising a cell.

10. The composition of claim 9, wherein the cell is an induced pluripotent stem cell.

11. The composition of claim 9, wherein the cell is a disease specific model cell.

12. A method of fabricating cell-laden hydrogel constructs comprising
a pH buffered liquid aqueous solution of cells comprising;
a photoinitiator;
methacrylated polyethylene glycol; and
methacrylated gelatin; and
exposing a zone within the liquid solution to light under conditions that polymerization provides a cell-laden hydrogel construct within the zone.

13. The method of claim 12, wherein the methacrylated polyethylene glycol is 90-95% functionalized.

14. The method of claim 12, wherein the methacrylated gelatin is 60-75% functionalized.

15. The method of claim 12, wherein the gelatin is cold-water fish gelatin optionally further comprising porcine gelatin.

16. The method of claim 12, wherein the methacrylated gelatin polymer is between 20-5% by weight to volume of the liquid.

17. The method of claim 12, wherein the photoinitiator is lithium phenyl-2,4,6-trimethylbenzoylphosphinate (LAP).

18. The method of claim 12, wherein the pH buffered liquid aqueous solution further comprises tartrazine.

19. The method of claim 12, wherein the cells are induced pluripotent stem cells.

20. The method of claim 12, wherein the cells are disease specific model cells.
